(19) United States
(12) Patent Application Publication
(10) Pub. No.: US 2023/0204476 A1
(43) Pub. Date: Jun. 29, 2023

(54) MERCURY VAPOR REFERENCE

(71) Applicant: AMETEK, INC., Berwyn, PA (US)

(72) Inventors: Archibald Williams, Wareham, MA (US); Alex R. Locascio, Phoenix, AZ (US); James A. Salomon, Providence, RI (US)

(21) Appl. No.: 18/085,762

(22) Filed: Dec. 21, 2022

Related U.S. Application Data
(60) Provisional application No. 63/293,467, filed on Dec. 23, 2021.

(51) Int. Cl. G01N 1/38; B01L 3/12

(52) U.S. Cl. CPC G01N 1/38; B01L 3/12; G01N 2001/382; B01L 2300/0861; B01L 2300/0627; B01L 2300/10; B01L 2300/049

(57) ABSTRACT

Mercury vapor reference systems, devices, and method for testing mercury vapor analyzers. A variable volume dilution chamber such as a syringe selectively receives mercury vapor and a dilution gas to dilute the mercury vapor prior to dispensing into an airflow for testing by the mercury vapor analyzer.

This application claims priority to U.S. Provisional Patent Application No. 63/293,467 titled MERCURY VAPOR REFERENCE (Atty. Docket No.: AMT-105USP), filed on Dec. 23, 2021, the contents of which are fully incorporated herein by reference.

### TECHNICAL FIELD

The present disclosure relates to mercury vapor analyzers and more particularly to mercury vapor references for use in testing mercury vapor analyzers.

### BACKGROUND

Mercury vapor analyzers are used to measure the concentration of mercury vapor in an environment. A mercury vapor analyzer such as the Jerome® J505 Mercury Vapor Analyzer (the J505) available from AMETEK Brookfield of Chandler, Ariz., measures the concentration in a continuous stream of air flowing through a chamber in the analyzer, which is illuminated by a mercury lamp and monitored with a photomultiplier tube, to determine the amount of mercury in the chamber. A mercury vapor reference is useful to confirm the accuracy of such mercury vapor analyzers.

### BRIEF DESCRIPTION OF THE DRAWINGS

The disclosed subject matter is best understood from the following detailed description when read in connection with the accompanying drawings, with like elements having the same reference numerals. When a plurality of similar elements is present, a single reference numeral may be assigned to the plurality of similar elements with a letter designation referring to specific elements. When referring to the elements collectively or to a non-specific one or more of the elements, the letter designation may be dropped. To easily identify the discussion of any particular element or act, the most significant digit or digits in a reference number refer to the figure number in which that element is first introduced. This emphasizes that according to common practice, the various features of the drawings are not drawn to scale unless otherwise indicated. On the contrary, the dimensions of the various features may be expanded or reduced for clarity. Included in the drawings are the following figures:

FIG. 1 is a block diagram of an example system including a mercury vapor analyzer and a mercury vapor reference for generating a reference mercury vapor for confirming the accuracy of the mercury vapor analyzer;

FIG. 2 is a block diagram of an example mercury vapor reference;

FIGS. 3A and 3B are illustrations depicting to example mixing chambers for use within the mercury vapor reference of FIG. 2;

FIG. 4A is a schematic diagram of an example mercury vapor reference;

FIG. 4B is a schematic diagram of an optional valve for controlling mercury flow positioned in the example mercury vapor reference of FIG. 4A;

FIGS. 5A, 5B, 5C, and 5D depict flow charts 500/520/540/550 of example methods for testing mercury vapor, determining whether a mercury vapor test is needed, initiating a mercury vapor test, and communicating with a mercury vapor analyzer, respectively;

FIG. 6 is a functional block diagram illustrating a general-purpose computer hardware platform configured to implement the functional examples described with respect to FIGS. 1-5; and

FIG. 7 is another functional block diagram illustrating a general-purpose computer hardware platform configured to implement the functional examples described with respect to FIGS. 1-5.

### DETAILED DESCRIPTION

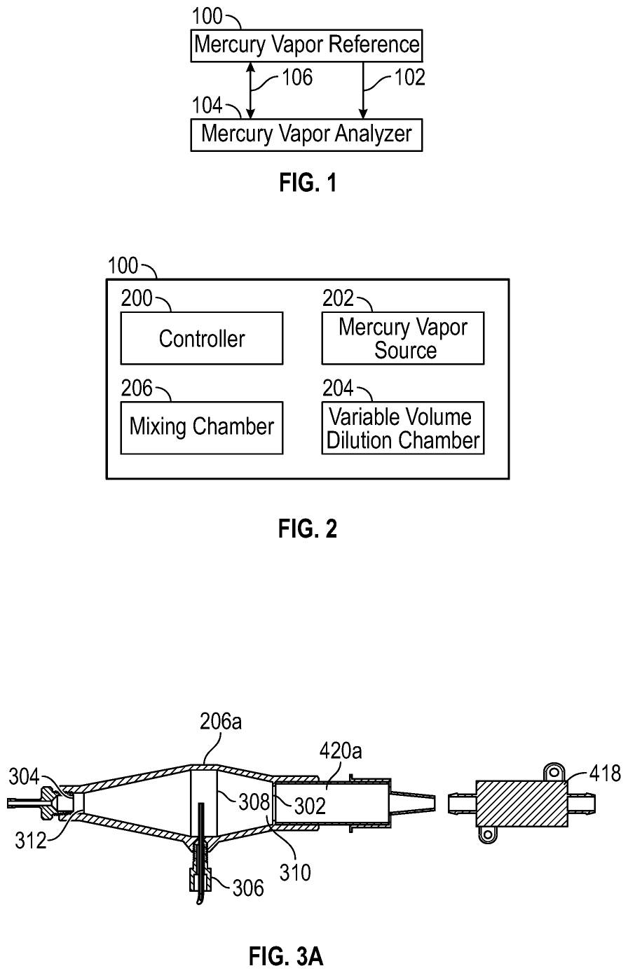

FIG. 1 depicts a mercury vapor reference 100 that produces a reference mercury vapor in a supply tube 102 for testing a mercury vapor analyzer 104. In one example, a data connection 106 is present between the mercury vapor reference 100 and the mercury vapor analyzer 104. The mercury vapor reference 100 may control the mercury vapor analyzer 104 via the data connection, write data to a memory or the mercury vapor analyzer 104, or a combination thereof. In one example, the mercury vapor analyzer 104 is the J505. The J505 draws air into a chamber using a pump (not shown) contained within the J505, thereby generating airflow.

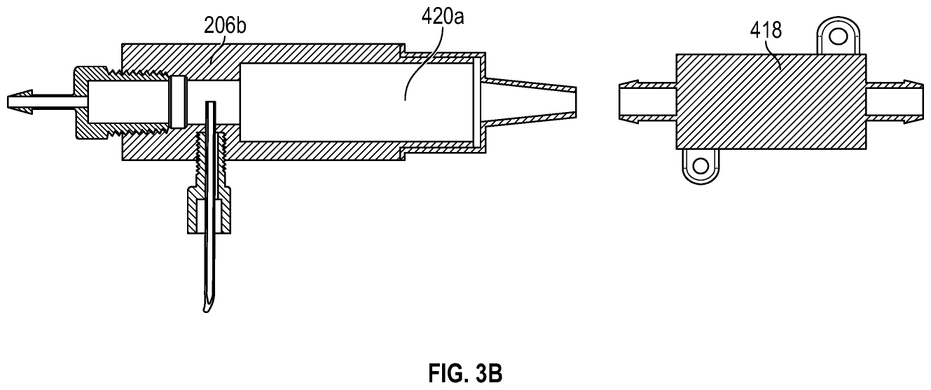

FIG. 2 depicts an example of components of the mercury vapor reference 100. The mercury vapor reference 100 includes a controller 200, a mercury vapor source 202 that produces mercury vapor, a variable volume dilution chamber 204 in which mercury vapor from the mercury vapor source 202 is initially diluted, and a mixing chamber 206 that introduces the diluted mercury vapor from the variable volume dilution chamber 204 to an airflow to generate a reference mercury vapor.

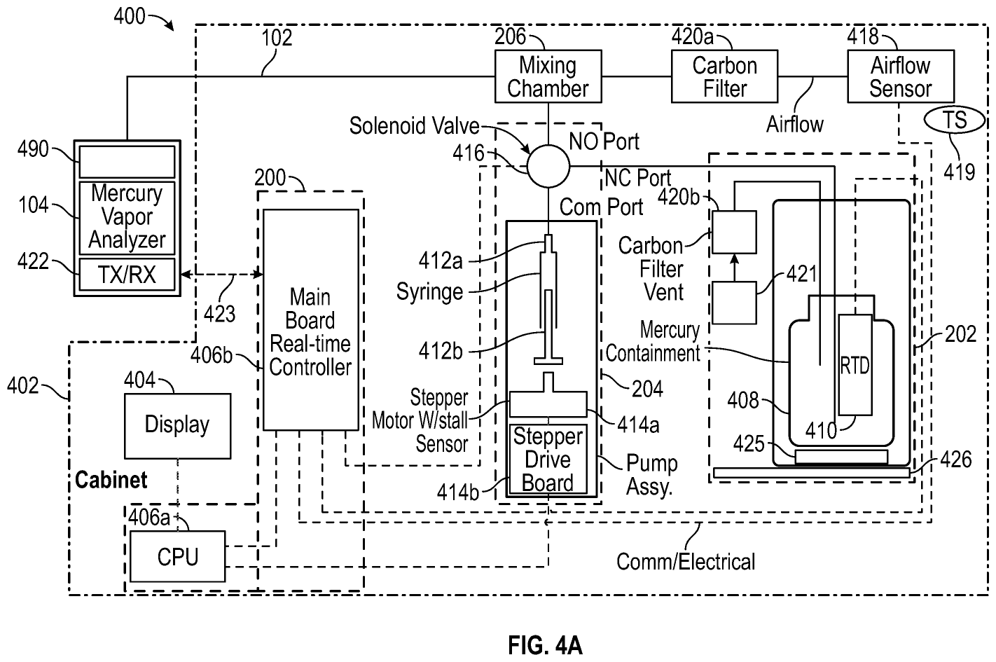

FIG. 3A depicts one example of a mixing chamber 206a for use in the mercury vapor reference 100. The illustrated mixing chamber 206a has a cylindrical airflow chamber with a radius that varies in the longitudinal direction. The mixing chamber 206a includes an airflow input 302 on a first end and an airflow output 304 on a second end. A mercury vapor input port 306 is located between the airflow input 302 the airflow output 304. The mixing chamber 206a has a greater cross-sectional area 308 adjacent the mercury vapor input port 306 than the input cross-sectional area 310 adjacent the airflow input 302 and the output cross-sectional area 312 adjacent the airflow output 304. In accordance with this example, the mixing chamber may have a volume of approximately 35 ml. FIG. 3B depicts another example of a mixing chamber 206 for use in the mercury vapor reference 100. The illustrated mixing chamber 206b has a cylindrical airflow chamber with a constant radius in the longitudinal direction. In accordance with other examples, the variable volume dilution chamber 204 may be connected directly to the supply tube 102 and the mixing chamber 206 may be omitted.

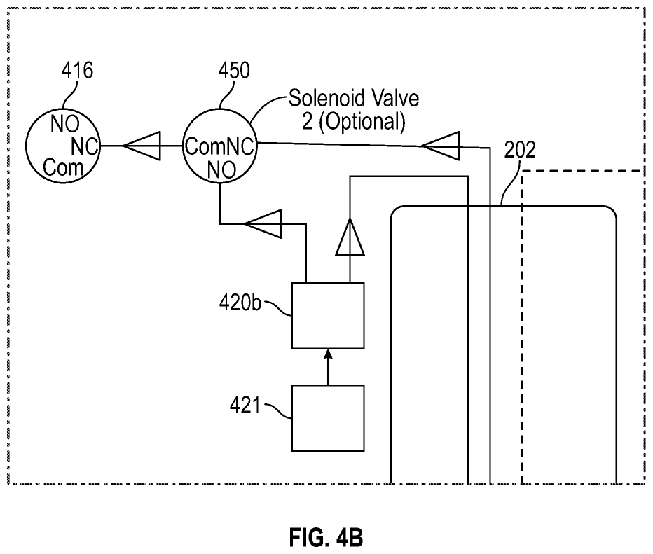

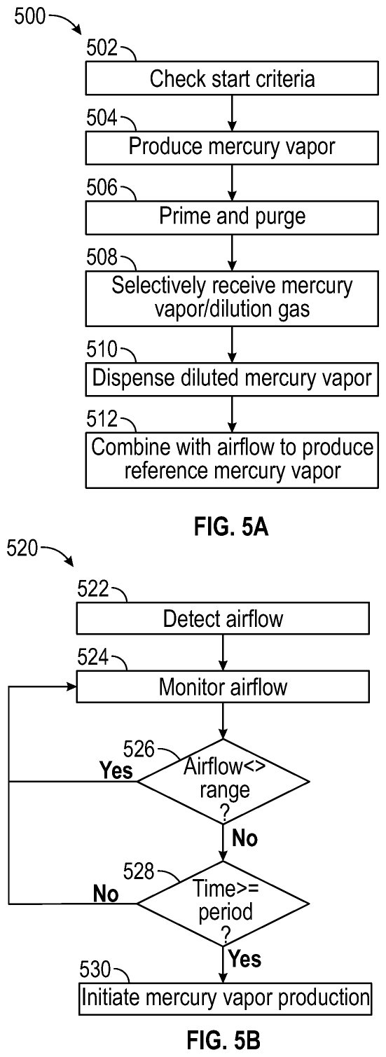

FIG. 4A depicts details of an example mercury vapor reference 400 for providing a reference mercury vapor in an airflow within the supply tube 102 for delivery to the mercury vapor analyzer 104. The mercury vapor reference 400 includes a cabinet 402 housing the controller 200, mercury vapor source 202, variable volume dilution chamber 204, and mixing chamber 206. The mercury vapor reference 400 additionally includes a display 404 for presenting information to a user and an input device such as a keypad/keyboard (not shown; which may be incorporated into the display 404).

The controller 200 in the mercury vapor reference 400 includes a central processing unit (CPU) 406a and an input/output (IO) board 406b (e.g., a VPXL main board real-time controller; VPXL) in communication with the CPU 406a. The CPU 406a is coupled directly to the display 404 and the components of the variable volume dilution chamber 204 and indirectly (via the IO board 406b) to the mercury vapor source 202, an airflow sensor 418 that senses airflow into the mixing chamber 206, and other components of the variable volume dilution chamber 204. The illustrated mercury vapor reference 400 additionally includes a temperature sensor 419 such as a thermocouple (e.g., for use in determining the density of air in the airflow entering the mixing chamber when calculating mercury vapor exiting the mixing chamber). The CPU 406a may additionally include a wired or wireless connection (not shown) to a controller 490 of the mercury vapor analyzer 104 for controlling operation of the mercury vapor analyzer 104 (e.g., during a testing phase), for writing data to the mercury vapor analyzer 104 (e.g., test results in a log stored in memory), or a combination thereof.

The mercury vapor reference 100 may be coupled to the mercury vapor analyzer 104 via a communication link 423. The communication link 423 may be a wired (e.g., USB) or wireless (e.g., Wi-Fi) link.) The mercury vapor analyzer 104 may include a transceiver 422 and the mercury vapor reference 100 may include a corresponding transceiver (not shown) for communication there between.

The mercury vapor source 202 in the mercury vapor reference 400 includes liquid mercury (not shown) within a container 408 and a temperature sensor 410 such as a resistance temperature detector (RTD). The mercury vapor source 202 may additionally include optional heating and/or cooling element(s) (not shown) for controlling the amount of mercury vapor (under control of the controller 200) delivered to the variable volume dilution chamber 204 if, for example, room temperature varies too much or too quickly to get a stable mercury vapor supply. The heating/cooling element(s), if incorporated, could be used to maintain a stable temperature, and could be located in the container 408 of the mercury vapor source 202. In one example, the container 408 has a volume of approximately 500 ml. A desiccant may be added to the container 408 to limit moisture within the mercury vapor reference 400.

Mercury concentration in air is linked to temperature by the Antoine equation, ideal gas law, and Dalton's law (see below), which are implemented by controller 200 to precisely control the concentration of mercury vapor delivered to the mercury vapor analyzer 104.

The Antoine Equation (e.g., to calculate the mercury vapor pressure):

log₁₀(P)=A−(B/(T+C))
where:
   P=vapor pressure (bar)
   T=temperature (298.14-749.99 K)
   A=8.274427
   B=3280.205
   C=273

Ideal Gas Law (e.g., to determine the # of molecules in a cubic meter):

n=(P*V)/(R*T)
where:
   n=moles
   P=pressure
   V=volume
   R=gas constant
   T=temperature

Dalton's Law (e.g., to find the actual mercury vapor concentration in the reservoir):

Pₜₒₜₐₗ=P₁+P₂+P₃+ . . . +Pₙ
where:
   Ptotal is the total pressure exerted by the mixture of gases
   P1, P2, . . . , Pn are the partial pressures of the gases 1, 2, . . . , ‘n’ in the mixture of ‘n’ gases

The variable volume dilution chamber 204 in the mercury vapor reference 400 includes a syringe having a barrel 412a and a piston 412b inserted into the barrel, wherein movement of the piston 412b relative to the barrel 412a changes the volume of the variable volume dilution chamber 204. In one example, the syringe has of volume of 5 ml, 10 ml syringe, or greater. It is contemplated that other types of variable volume dilution chambers 204 may be used. For example, the variable volume dilution chamber, may be a baffled cylinder that is expanded/contracted to change the volume or a rectangular box with one or more flexible side walls that deform(s) outward/inward to change the volume. Other ways of changing the volume will be readily apparent to those skilled in the art from the description herein and are to be considered within the scope of the present disclosure.

The variable volume dilution chamber 204 illustrated in FIG. 4A further includes a stepper motor 414a configured to move the piston 412b within the barrel 412a to change the volume of the variable volume dilution chamber 204. Movement of the stepper motor 414a is implemented through a stepper drive board 414b under control of the controller 200.

The variable volume dilution chamber 204 illustrated in FIG. 4A additionally includes a valve 416. In the illustrated example, the valve 416 is a solenoid valve having a common port coupled to an output of the barrel 412a, a normally closed (NC) port coupled to an output of the mercury vapor source 202, and a normally open (NO) port coupled to an input of the mixing chamber 206. Although a single valve is depicted, multiple valves may be used (e.g., a separate valve connected between the output of the syringe and each of the mercury vapor source 202 and the mixing chamber 206.

The mixing chamber 206 in the mercury vapor reference 400 includes a cylindrical airflow chamber. An airflow input port is positioned on one end of the cylindrical airflow chamber and an airflow output port is positioned on the other end of the cylindrical airflow chamber. A mercury vapor input into the cylindrical airflow chamber is located between the airflow input and the airflow output. In an example, the cylindrical airflow chamber has a greater cross-sectional area adjacent the mercury vapor input port than adjacent the airflow input and output ports. This arrangement reduces the speed of air flowing past the mercury vapor input port to reduce mercury vapor being drawn into the mixing chamber due to the air flow past the port, which allows the control of the mercury vapor into the mixing chamber to be controlled primarily by changing the volume of the variable volume dilution chamber 204.

The mercury vapor reference 400 may additionally include one or more of a mixer for vapor mixing/stirring in a mercury source container (e.g., to expose fresh surfaces), elemental (liquid) mercury containment in semi permeable containers (e.g., a membrane) to limit movement in the mercury source container, baffles in the mixing chamber to mix mercury/air (e.g., screen or beads in flow path, or an additional valve to self-check mercury containment (e.g., draw air from secondary containment directly to the mercury analyzer such as the J505).

The mercury vapor reference 400 additionally includes an agitator and/or heat exchanger 425. The agitator is configured to periodically agitate the mercury vapor source 202 (e.g., upon startup, every 24 hours, etc.), which has been discovered to renew (e.g., reduce/eliminate effects of tarnish when mercury remains still) the mercury vapor source 202 for delivering mercury vapor. In one example, the agitator includes a rocker that rocks the container 408 (e.g., plus/minus 30 degrees). In another example, the agitator includes a stirrer on an outside of the container 408 that is magnetically couple to a stir rod on the inside of the container 408, which it rotated by the stirrer. In another example, the agitator is a sonic agitator such as a voice coil. The heat exchanger may be a cold plate/coil of tubing for use in controlling the temperature of the mercury in the container 408.

The mercury vapor reference 400 additionally includes a mercury leak detector and/or mercury containment system 426. The mercury leak detector is configured to detect mercury leaks. In one example, the leak detector is a color change material that changes color in the presence of mercury. In accordance with this example, the color change material may be positioned at seams of the cabinet 402 and around openings of the cabinet 402 to alert a user that mercury is present. The mercury containment system is configured to contain leaking mercury within the cabinet 402. In one example, the mercury containment system includes one or more packets of mercury absorbing material (e.g., zinc oxide powder). In another example, the mercury containment system is an air permeable mercury phobic membrane that encapsulates the container 408.

FIG. 4B depicts an optional valve 450 for controlling mercury flow in the mercury vapor reference 400 (FIG. 4A). The valve 450 is positioned between the mercury vapor source 202 and the valve 416 of the variable volume dilution chamber 204. This arrangement avoids recirculating trace mercury to the variable volume dilution chamber 204. In this arrangement, the normally closed port of the valve 450 is only open when the variable volume dilution chamber 204 is expanding and the normally open port of the valve 416 is closed (i.e., creating a vacuum in the system withdrawing mercury vapor from the mercury vapor source 202. Air free from trace mercury for clearing the tubing and valves from trace mercury vapor is drawn through the normally open port of the valve 450 and a carbon filter 420b. A desiccant 421 may be positioned before or after the carbon filter 420b to prevent water vapor from entering the container 408 of the mercury vapor source 202 so that dry air is delivered to the container 408.

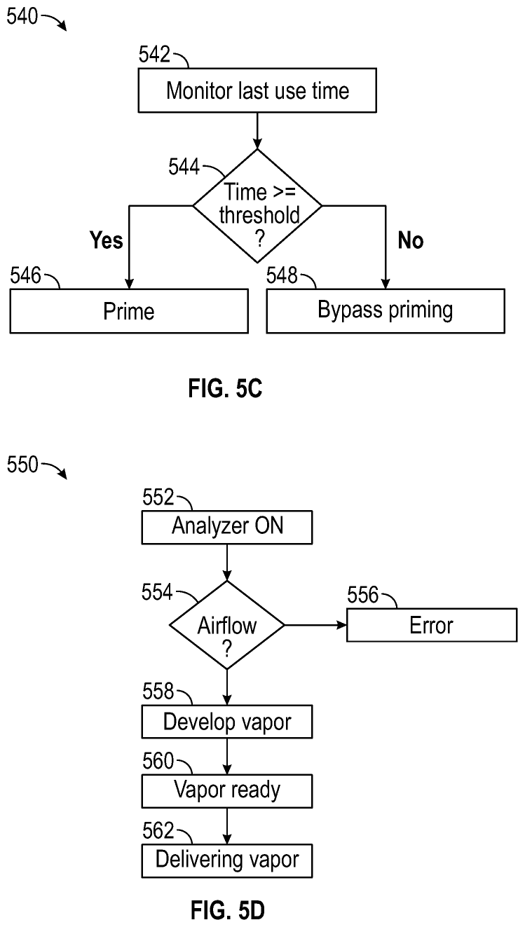

FIGS. 5A, 5B, 5C, and 5D depict flow charts 500/520/540/550 of example methods for producing reference mercury vapor to test a mercury vapor analyzer, determining when to initiate production of mercury vapor, determining when to bypass priming when producing reference mercury vapor, and communicating with a mercury vapor analyzer, respectively. These steps are performed to produce a reference mercury vapor to verify sensitivity of a mercury vapor analyzer prior to starting an environmental assessment. Although the steps are described with reference to the mercury vapor reference 400 described herein, other implementations of the steps described, for other types of devices, will be understood by one of skill in the art from the description herein. One or more of the steps shown and described may be performed simultaneously, in a series, in an order other than shown and described, or in conjunction with additional steps. Some steps may be omitted or, in some applications, repeated.

At block 502 (FIG. 5A), the mercury vapor reference 400 checks starting criteria. In an example, the starting criteria is used to determine if a mercury vapor analyzer 104 is operating. During operation in accordance with this example, the mercury vapor analyzer 104 draws air into a chamber of the analyzer using a pump. When connected to a supply tube 102, the mercury vapor analyzer 104 generates airflow through the mixing chamber 206.

One example for checking starting criteria is depicted in FIG. 5B. An airflow sensor 418 detects airflow entering the mixing chamber and flowing to the mercury vapor analyzer 104 through the supply tube 102 (block 522). A controller 200 coupled to the airflow sensor 418 monitors the airflow (block 524). The controller 200 checks whether the airflow is outside a predefined range (e.g., a range associated with proper operation of the mercury vapor analyzer 104; block 526). If the airflow is outside the predefined range, the controller 200 continues to monitor the airflow. If the airflow is within the predefined range, the controller 200 increments a clock value and determines if the clock value is equal to or greater than a predefined period of time (e.g., 15 seconds; block 528). If the clock value indicates the predefined period of time has not been reached, the controller 200 continues to monitor airflow and increments the clock value while the airflow is within the predefined range. If the clock value indicates the predefined period of time has been reached, the controller 200 determines that the starting criteria has been met and initiates mercury vapor productions (block 530).

In one example, airflow is continuously monitored during operation of the mercury vapor reference 100 as described with reference to blocks 522-526. In accordance with this example, if airflow stops or is out of the range, the mercury vapor reference 100 stops delivering mercury vapor (e.g., by halting movement of piston 412b). A piston position detector (not shown) may monitor position of the piston for use in restarting the delivery of mercury vapor when suitable airflow is detected after halting delivery of mercury vapor.

In one example, at startup, the connection lines and storage spaces (e.g., like a syringe) are cleaned and/or primed so that the concentration of mercury vapor in the lines/spaces are known. For example, the feed line from the mercury vapor source to the valve—about 1 mL of tubing volume—may contain a low percentage of mercury when the system has been idle for a long period (e.g., two hours). The line may be purged to ensure full potency of the vapor contained in the mercury flask. Additionally, the injector line from the valve to the mixing chamber—about 0.1 mL—may contain a mixture of air/mercury at the end of each cycle. Further, the syringe=about 5 to 10 mL—may be emptied on powerup and after each cycle.

At block 504 (FIG. 5A), the mercury vapor reference produces mercury vapor. In an example, the mercury vapor source 202 produces the mercury vapor. In accordance with this example, mercury in the container 408 is temperature controlled by the CPU 406a responsive to feedback from the temperature sensor 410. By controlling the temperature of the mercury, the concentration of mercury vapor in the container 408 can be precisely controlled.

At block 506, the mercury vapor reference is primed and purged using the variable volume dilution chamber 204 (e.g., if the mercury vapor reference has not been used for a predefined period of time). In an example, the mercury vapor reference is primed and purged using a syringe as the variable volume dilution chamber. In accordance with this example, to prime the mercury vapor reference, the normally open port is closed, the normally closed port is open, and the piston 412b of the syringe is withdrawn from the barrel 412a (e.g., starting at 0 volume)—thereby increasing its volume to create a vacuum drawing mercury vapor from the mercury vapor source 202 through the normally closed port of the valve 416 into the barrel of the syringe. Then, the normally open port is open, the normally closed port is closed (with full potency mercury trapped in the feed line), and the piston 412b of the syringe is inserted into the barrel 412a—thereby decreasing its volume to deliver mercury vapor through the valve 416 and associated connection components into the mixing chamber 206. To purge the mercury vapor reference, the normally open port remains open, the normally closed port remains closed, and the piston 412b of the syringe is first withdrawn from the barrel 412a—thereby increasing its volume to create a vacuum drawing residual mercury vapor from the mixing chamber through the valve 416 and associated connection components into the barrel of the syringe; and then is fully inserted. The ratio of clean air to vapor should be high enough to prevent changing the final mix.

At block 508, the mercury vapor reference selectively receives the mercury vapor and a dilution gas (e.g., air or filtered air). The mercury vapor is withdrawn from the mercury vapor source 202 and the dilution gas is withdrawn from the environment (e.g., via the mixing chamber 206). In one example, the mercury vapor analyzer 104 includes a pump that draws air into a chamber of the analyzer. In accordance with this example, the airflow passing through the mixing chamber is a result of the pump in the mercury vapor analyzer 104. A carbon filter 420a may be positioned at an input port of the mixing chamber 206 to filter out mercury and other contaminants that may be present in the air. Another carbon filter 420b and/or a desiccant 421 may be positioned at an input to the container 408 of the mercury vapor source 202 to remove trace mercury from the environment and/or water vapor when air is drawn into the container 408 (e.g., when the variable volume dilution chamber 204 expands with the normally closed port of the valve 416 open).

In an example, the syringe of the variable volume dilution chamber 204 selectively receives the mercury vapor and the dilution gas. In accordance with this example, the piston 412b of the syringe is withdrawn from the barrel 412a—thereby increasing its volume to create a vacuum drawing air from the airflow passing through the mixing chamber 206 and the normally open port of the valve 416 into the barrel of the syringe. Next, the normally open port is closed, the normally closed port is open, and the piston 412b of the syringe is further withdrawn from the barrel 412a—thereby further increasing its volume to create a vacuum drawing mercury vapor from the mercury vapor source 202 through the normally closed port of the valve 416 into the barrel of the syringe. Then, the normally closed port is closed, the normally open port is open, and the piston 412b of the syringe is further withdrawn from the barrel 412a—thereby further increasing its volume to create a vacuum drawing additional air from the airflow passing through the mixing chamber 206 and the normally open port of the valve 416 into the barrel of the syringe. This three-step sequential process is controlled by the CPU 406a, which controls the amount the piston is withdrawn from the barrel during each step, to precisely control the concentration of mercury in the now diluted mercury vapor within the variable volume dilution chamber 204. In alternative arrangements, only two sequential steps may be performed (e.g., one for air and one for mercury vapor) or more than three alternating steps may be performed to obtain the desired concentration of mercury vapor in the diluted mercury vapor.

At block 510, the mercury vapor reference dispenses the diluted mercury vapor. In an example, after the desired concentration of mercury vapor in the diluted mercury vapor is achieved, the variable volume dilution chamber 204 dispenses the diluted mercury vapor. In accordance with this example, the piston 412b of the syringe is inserted into the barrel 412a—thereby decreasing its volume to force the diluted mercury vapor through the normally open port of the valve 416 into the mixing chamber 206 (e.g., via mercury vapor input port).

At block 512, the mercury vapor reference combines the diluted mercury vapor with the airflow to produce the reference mercury vapor for testing the mercury vapor analyzer 104. In an example, the diluted mercury vapor is combined with the airflow in the mixing chamber 206.

The controller 200 controls the concentration of mercury in the mercury vapor source, the amount of dilution to create the diluted mercury vapor, and the rate of delivery to the airflow in the mixing chamber to produce the reference mercury vapor in the airflow traveling through the supply tube 102 for measurement by the mercury vapor analyzer during a testing phase. In one example, the rate of insertion is based on at least the potency of the mercury from the mercury vapor source (Antoine equation). The controller 200 may additionally control the temperature of the mercury container via a heat exchanger and obtain readings from the temperature sensor 419 to determine the density of the airflow for use in determining the appropriate amount of mercury vapor to add to produce airflow with the desired reference mercury vapor.

The concentration of mercury in the reference mercury vapor may be compared by the controller 200 to results measured by the mercury vapor analyzer 104 and the controller 200 may store the supplied concentrations, test results, and a time stamp via a data connection in a log within the mercury vapor analyzer 104. The controller 200 may additionally calculate and store number of cycles run and maintenance intervals.

At block 542 (FIG. 5C), the mercury vapor reference monitors a time since it was last used. In an example, the controller 200 initiates a timer in response to the controller 200 halting dispensing of diluted mercury vapor by the variable volume dilution chamber 204.

At decision block 544, the mercury vapor reference compares the elapsed time since it was last used to a threshold (e.g., 2 hours). In an example, the controller 200 compares a current elapsed time of a timer to the threshold. If the current elapsed time is greater than or equal to the threshold, priming/purging (block 506) is performed (block 546). If the current elapsed time is less than the threshold, priming/purging is not performed (block 548). Additionally, the mercury vapor reference may compare the elapsed time to another threshold (e.g., 24 hours). If the current elapsed time is greater than or equal to this other threshold, the mercury vapor source is agitated using an agitator controlled by the controller 200.

At block 552 (FIG. 5D), the mercury vapor reference determines is the mercury vapor analyzer is on. The mercury vapor reference 100 may determine if the mercury vapor analyzer 104 is on via the communication link. To determine if the mercury vapor analyzer 104 is one, the mercury vapor reference 100 may periodically query the mercury vapor analyzer 104 or may monitor a channel/port for a communication from the mercury vapor analyzer 104 indicating that the mercury vapor analyzer is on.

At decision block 554, after the mercury vapor reference 100 determines that the mercury vapor analyzer 104 is on, the mercury vapor reference 100 detects whether or not there is airflow. The presence/absence of airflow may be detected as described herein with reference to blocks 522 and 524. If airflow is not detected, the mercury vapor reference 100 may generate an error at block 556 for presentation (audible and/or visual) by mercury vapor reference 100 or for communication to (e.g., via link 423) and presentation by mercury vapor analyzer 104.

At block 558, when airflow is detected, the mercury vapor reference 100 begins developing mercury vapor. The development of mercury vapor may be performed as described herein with reference to block 504.

At block 560, the mercury vapor reference determines that the developed mercury vapor is ready for delivery to the airflow. In one example, the mercury vapor reference 100 communicates to the mercury vapor analyzer 104 that the mercury vapor is ready for delivery to the airflow, which may prompt the mercury vapor analyzer 104 to initiate entry into a testing phase.

At block 562, the mercury vapor reference begins delivering mercury vapor to the airflow. In one example, the mercury vapor reference 100 communicates to the mercury vapor analyzer 104 that the mercury vapor is being delivered to the airflow, which may prompt the mercury vapor analyzer 104 to begin recording mercury vapor levels in the airflow. In this manner, the mercury vapor reference 100 and mercury vapor analyzer 104 can coordinate mercury vapor delivery/testing to align the testing to the delivery of the mercury vapor.

In use, when used with the J505, the J505 measures the concentration of mercury in a continuous stream of air flowing through a chamber in the analyzer which is illuminated by a mercury lamp and monitored with a photomultiplier tube, to determine the amount of mercury in the chamber. The mercury vapor reference 100 described herein can be used to create a reference mercury vapor for confirming the accuracy of the mercury vapor analyzer.

The J505 is approved for testing mercury vapor concentrations of 1 micro gm/cu·m. To be used regularly for this purpose, users are seeking a dependable mercury vapor reference source that can easily generate this threshold level. Using a mercury vapor reference according to aspect described herein, a user can check their J505 daily, to be sure that the system sensitivity is within range before starting an environmental assessment.

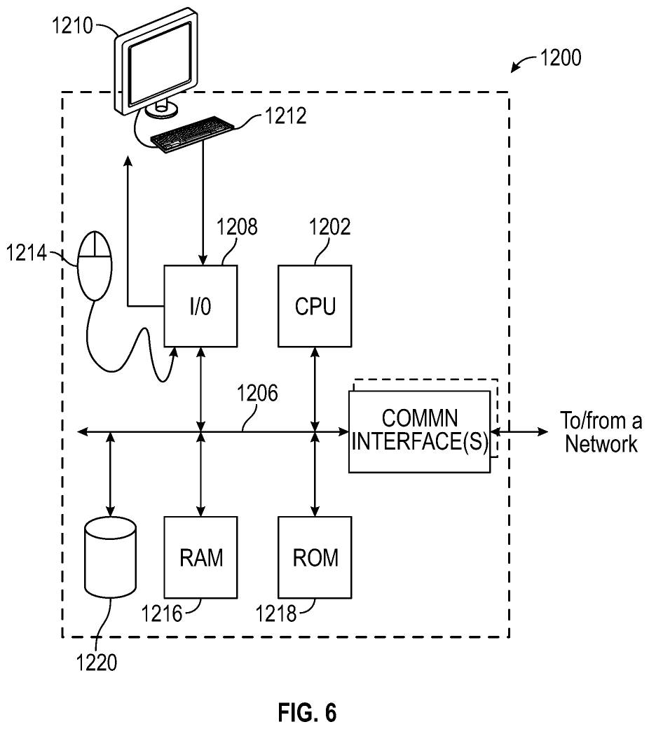

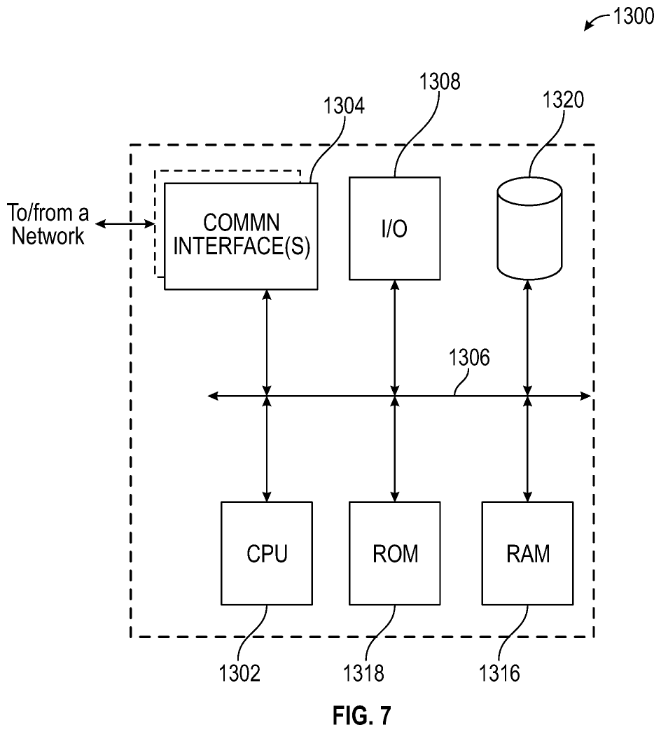

FIGS. 6 and 7 are functional block diagrams illustrating general-purpose computer hardware platforms configured to implement the functional examples described with respect to FIGS. 1-5 as discussed above.

Specifically, FIG. 6 illustrates an example network or host computer platform 1200, as may be used to implement for implementing a server. Specifically, FIG. 7 depicts an example computer 1300 with user interface elements, as may be used to implement a personal computer or other type of workstation or terminal device, although the computer 1300 of FIG. 7 may also act as a server if appropriately programmed. It is believed that those skilled in the art are familiar with the structure, programming, and general operation of such computer equipment and as a result the drawings should be self-explanatory.

Hardware of an example server computer (FIG. 6) includes a data communication interface for packet data communication. The server computer also includes a central processing unit (CPU) 1202, in the form of circuitry forming one or more processors, for executing program instructions. The server platform hardware typically includes an internal communication bus 1206, program and/or data storage 1216, 1218, and 1220 for various programs and data files to be processed and/or communicated by the server computer, although the server computer often receives programming and data via network communications. In one example, as shown in FIG. 6, the computer system includes a video display unit 1210, (e.g., a liquid crystal display (LCD) or a cathode ray tube (CRT)), an alphanumeric input device 1212 (e.g., a keyboard), a cursor control device 1214 (e.g., a mouse), each of which communicate via an input/output device (I/O) 1208. The hardware elements, operating systems and programming languages of such server computers are conventional in nature, and it is presumed that those skilled in the art are adequately familiar therewith. Of course, the server functions may be implemented in a distributed fashion on a number of similar hardware platforms, to distribute the processing load.

Hardware of a computer type user terminal device, such as a PC or tablet computer, similarly includes a data communication interface 1304, CPU 1302, main memory 1316 and 1318, one or more mass storage devices 1320 for storing user data and the various executable programs, an internal communication bus 1306, and an input/output device (I/O) 1308 (see FIG. 7).

Aspects of this disclosure, as outlined above, may be embodied in programming in general purpose computer hardware platforms (such as described above with respect to FIGS. 6 and 7), e.g., in the form of software, firmware, or microcode executable by a computer system such as a server or gateway, and/or a programmable nodal device. Program aspects of the technology may be thought of as “products” or “articles of manufacture” typically in the form of executable code and/or associated data that is carried on or embodied in a type of machine readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software, from one computer or processor into another. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to one or more of “non-transitory,” “tangible” or “storage” media, terms such as computer or machine “readable medium” refer to any medium that participates in providing instructions to a processor for execution.

Aspects of the methods of this disclosure, as outlined above, may be embodied in programming in general purpose computer hardware platforms (such as described above with respect to FIGS. 6 and 7), e.g., in the form of software, firmware, or microcode executable by a networked computer system such as a server or gateway, and/or a programmable nodal device. Program aspects of the technology may be thought of as “products” or “articles of manufacture” typically in the form of executable code and/or associated data that is carried on or embodied in a type of machine readable medium. “storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software, from one computer or processor into another. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to one or more of “non-transitory,” “tangible” or “storage” media, terms such as computer or machine “readable medium” refer to any medium that participates in providing instructions to a processor for execution.

Hence, a machine-readable medium may take many forms, including but not limited to, a tangible storage medium, a carrier wave medium or physical transmission medium. Non-transitory storage media include, for example, optical or magnetic disks, such as any of the storage devices in any computer(s) or the like. It may also include storage media such as dynamic memory, for example, the main memory of a machine or computer platform. Tangible transmission media include coaxial cables; copper wire and fiber optics, including the wires that include a bus within a computer system. Carrier-wave transmission media can take the form of electric or electromagnetic signals, or acoustic or light waves such as those generated during radio frequency (RF) and light-based data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media may be involved in carrying one or more sequences of one or more instructions to a processor for execution.

Program instructions may include a software or firmware implementation encoded in any desired language. Programming instructions, when embodied in machine readable medium accessible to a processor of a computer system or device, render computer system or device into a special-purpose machine that is customized to perform the operations specified in the program performed by electronics of the mercury vapor reference 100.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of sections 101, 102, or 105 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that includes a list of elements does not include only those elements but may include other elements not expressly listed or inherent to such process, method, article, or apparatus. An element preceded by “a” or “an” does not, without further constraints, preclude the existence of additional identical elements in the process, method, article, or apparatus that includes the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than all features of any single disclosed example. Thus, the following claims are hereby incorporated into the Detailed Description, with each claim standing on its own as a separately claimed subject matter.

These and other advantages of the present invention will be apparent to those skilled in the art from the foregoing specification. Accordingly, it is to be recognized by those skilled in the art that changes or modifications may be made to the above-described embodiments without departing from the broad inventive concepts of the invention. It is to be understood that this invention is not limited to the particular embodiments described herein, but is intended to include all changes and modifications that are within the scope and spirit of the invention.

What is claimed is:

1. A mercury vapor reference configured to deliver a reference mercury vapor for testing a mercury vapor analyzer, the mercury vapor reference comprising:
a mercury vapor source configured to produce mercury vapor;
a variable volume dilution chamber configured to selectively receive and dispense the mercury vapor from the mercury vapor source and a dilution gas, wherein the mercury vapor from the mercury vapor source is diluted with the dilution gas in the variable volume dilution chamber to produce a diluted mercury vapor prior to dispensing;
a mixing chamber having an airflow path and a mercury vapor inlet port configured to receive the diluted mercury vapor from the variable volume dilution chamber and introduce the received diluted mercury vapor to the mixing chamber; and
a controller coupled to the variable volume dilution chamber, the controller configured to control the variable volume dilution chamber to receive and dispense the mercury vapor and the dilution gas to produce the diluted mercury vapor, wherein the reference mercury vapor is produced in the airflow path when the diluted mercury vapor is introduced to the airflow path via the inlet port.

2. The mercury vapor reference of claim 1, wherein the variable volume dilution chamber comprises a syringe having a barrel and a piston inserted within the barrel and wherein the mercury vapor reference further comprises:
a stepper motor configured to received signals from the controller and to move the piston within the barrel to vary the volume of the variable volume dilution chamber responsive to the signals.

3. The mercury vapor reference of claim 2, wherein the syringe has an opening and wherein the variable volume dilution chamber further comprises:
a solenoid coupled to the controller, the solenoid having a common port coupled to the opening of the syringe, a normally open port coupled to the mercury vapor inlet port of the mixing chamber, and a normally closed port coupled to the mercury vapor source.

4. The mercury vapor reference of claim 1, wherein the mixing chamber has a cylindrical airflow chamber, an airflow input on a first end of the cylindrical airflow chamber, and an airflow output on a second end of the cylindrical airflow chamber, wherein the mercury vapor inlet port is located between the airflow input and the airflow output, and wherein the cylindrical airflow chamber has a greater cross-sectional area adjacent the mercury vapor input port than adjacent the airflow input and the airflow output.

5. The mercury vapor reference of claim 4, further comprising:
an airflow sensor coupled to controller and to the airflow input on the first end of the chamber, the airflow sensor configured to detect airflow into the mixing chamber and communicate the detected airflow to the controller.

6. The mercury vapor reference of claim 5, wherein the controller is configured to monitor the detected airflow, compare the detected airflow to a range corresponding to operation of the mercury vapor analyzer, and to initiate production of mercury by the mercury vapor source responsive to the detected airflow being within the range for at least a predefined period of time.

7. The mercury vapor reference of claim 1, wherein the controller comprises a data connection configured for communication with the mercury vapor analyzer and wherein the controller is configured to control the mercury vapor analyzer during a testing phase.

8. The mercury vapor reference of claim 1, further comprising:
a valve positioned between the variable volume dilution chamber, the mercury vapor source, and an air source;
wherein airflow to the mercury vapor inlet port of the mixing chamber is one way.

9. The mercury vapor reference of claim 1, wherein the controller is configured to:
prime the mercury vapor reference by drawing a concentrated mercury vapor from the mercury vapor source into the variable volume dilution chamber and dispensing the concentrated mercury vapor from the variable volume dilution chamber into the mixing chamber; and
purge residual mercury vapor from the mercury vapor reference by drawing air from the mixing chamber into the variable volume dilution chamber.

10. The mercury vapor reference of claim 1, further comprising:
a temperature sensor configured to sense temperature in the airflow path;
wherein the controller determines density of air in the airflow path responsive to the sensed temperature to produce the reference mercury vapor.

11. The mercury vapor reference of claim 1, further comprising at least one of:
an agitator configured to agitate the mercury vapor source;
a heat exchanger configured to control a temperature of the mercury vapor source;
a mercury detector configured to detect mercury leaks; or
a mercury containment system configured to contain mercury leaks.

12. The mercury vapor reference of claim 1, wherein the mercury vapor source has an inlet coupled to atmosphere and wherein the mercury vapor reference further comprises at least one of:
a filter coupled to the inlet of the mercury vapor source; or
a desiccant coupled to the inlet of the mercury vapor source.

13. A method for producing a reference mercury vapor to test a mercury vapor analyzer, the method comprising:
producing a mercury vapor with a mercury vapor source;
selectively receiving the mercury vapor from the mercury vapor source and a dilution gas in a variable volume dilution chamber to produce a diluted mercury vapor;
dispensing the diluted mercury vapor from the variable volume dilution chamber into an airflow path to produce the reference mercury vapor.

14. The method of claim 13, wherein the variable volume dilution chamber comprises a syringe having a barrel and a piston inserted within the barrel and wherein the selectively receiving and the dispensing comprises:
moving the piston within the barrel to vary the volume of the variable volume dilution chamber.

15. The method of claim 13, wherein the variable volume dilution chamber comprises a syringe having a barrel and a piston inserted within the barrel, the syringe has an opening, the variable volume dilution chamber further comprises a solenoid having a common port coupled to the opening of the syringe, a normally open port coupled to the airflow path, and a normally closed port coupled to the mercury vapor source, and the selectively receiving comprises:
withdrawing the piston from the barrel with the normally open port open to draw air into the variable volume dilution chamber from the airflow path during a first time period;
withdrawing the piston from the barrel with the normally closed port open to draw the mercury vapor into the variable volume dilution chamber during a second time period after the first time period;
withdrawing the piston from the barrel with the normally open port open to draw additional air into the variable volume dilution chamber during a third time period after the second time period to produce the diluted mercury vapor.

16. The method of claim 15, wherein the dispensing comprises:
inserting the piston into the barrel with the normally open port open to dispense the diluted mercury into the airflow to produce the reference mercury vapor.

17. The method of claim 13, wherein the dispensing comprises:
dispensing the diluted mercury into a mixing chamber having a cylindrical airflow chamber, an airflow input on a first end of the cylindrical airflow chamber, an airflow output on a second end of the cylindrical airflow chamber, and a mercury vapor input port configured to receive the diluted mercury located between the airflow input and the airflow output and wherein the cylindrical airflow chamber has a greater cross-sectional area adjacent the mercury vapor input port than adjacent the airflow input and the airflow output.

18. The method of claim 17, further comprising:
detecting airflow at least one of into or out of the mixing chamber;
monitoring the detected airflow;
comparing the detected airflow to a range corresponding to operation of the mercury vapor analyzer; and
initiating the selectively receiving the mercury vapor and the dilution gas when the detected airflow is within the range for at least a predefined period of time.

19. The method of claim 13, further comprising:
communicating with the mercury vapor analyzer; and
controlling the mercury vapor analyzer at least one of before or during a testing phase.

20. The method of claim 19, wherein the method further comprises:
gathering test parameters during the testing phase; and
recording the gathered test parameters to a log in a memory of the mercury vapor analyzer.

21. The method of claim 17, further comprising:
priming the mercury vapor reference by drawing a concentrated mercury vapor from the mercury vapor source into the variable volume dilution chamber and dispensing the concentrated mercury vapor from the variable volume dilution chamber into the mixing chamber; and
purging residual mercury vapor from the mercury vapor reference by drawing air from the mixing chamber into the variable volume dilution chamber.

22. The method of claim 21, further comprising:
monitoring a time since the mercury vapor reference was last used; and
bypassing the priming and purging if the monitored time is below a last use threshold.

23. The method of claim 13, further comprising:
cooling the mercury vapor source.

24. The method of claim 13, further comprising:
agitating the mercury vapor source to refresh the mercury vapor being produced.
